(19) United States
(12) Patent Application Publication
(10) Pub. No.: US 2022/0076835 A1
(43) Pub. Date: Mar. 10, 2022

(54) DISEASE JUDGEMENT METHOD

(71) Applicant: VUNO Inc., Seoul (KR)

(72) Inventors: Byeong Tak LEE, Seoul (KR); Woong BAE, Seoul (KR); Oyeon KWON, Seoul (KR)

(21) Appl. No.: 17/466,736

(22) Filed: Sep. 3, 2021

(30) Foreign Application Priority Data
Sep. 4, 2020 (KR) 10-2020-0113261

(51) Int. Cl. G16H 50/20; G06N 3/08; G06K 9/62

(52) U.S. Cl. CPC G16H 50/20; G06K 9/6267; G06N 3/08

(57) ABSTRACT

According to an embodiment of the present disclosure, disclosed is a computer program stored in a computer readable storage medium, in which when the computer program is executed on at least one processor, the computer program causes the processor to perform the following operations for judging a disease using a neural network, the operations including: acquiring one or more bio signals respectively measured in one or more leads; and generating result information about a disease by inputting the one or more bio signals into a disease judgment model.

### CROSS-REFERENCE TO RELATED APPLICATIONS

This application claims priority to and the benefit of Korean Patent Application No. 10-2020-0113261 filed in the Korean Intellectual Property Office on Sep. 4, 2020, the entire contents of which are incorporated herein by reference.

### BACKGROUND

### Technical Field

The present disclosure relates to a disease judgement method, and more particularly, to a disease judgement method using a neural network.

### Description of the Related Art

As chronic diseases (for example, cardiovascular disease and diabetes) increase and diversify in modern society, the need for continuous management is increasing.

Korean Patent No. 10-1799194 (Nov. 13, 2017) discloses an arrhythmia diagnosis device using an electrocardiogram signal.

### BRIEF SUMMARY

The inventors of the present disclosure have appreciated and recognized that existing equipment in the related art has a problem in continuous management because the existing equipment can be operated only in a hospital due to problems, such as volume and price. Further, since the existing device and method judge only one disease, in the case of a patient suffering from two or more chronic diseases, the numerical value is measured by using various equipment, and in this case, there is a problem that a lot of time and money is consumed.

The inventors of the present disclosure have provided one or more embodiments of a disease judgement method which addresses one or more problems in the related art as well as the technical problems identified by the inventors.

In order to solve the one or more problems in the related art, there is disclosed a computer program stored in a computer readable storage medium, in which wherein when the computer program is executed on at least one processor, the computer program causes the processor to perform the following operations for judging a disease using a neural network, the operations including: acquiring one or more bio signals respectively measured in one or more leads; and generating result information about a disease by inputting the one or more bio signals into a disease judgment model.

Alternatively, the disease judgment model may include: an encoding module comprising one or more encoding sub modules; a concatenation module for generating concatenation encoding data by concatenating one or more data encoded by the encoding module; and a classification module that receives the concatenation encoding data and generates the result information.

Alternatively, each of the one or more encoding sub modules may include a plurality of blocks that perform an encoding operation, and at least one block of the plurality of blocks may include a skip connection.

Alternatively, each of the one or more encoding sub modules may share at least some of weight.

Alternatively, the classification module may include one or more classification sub modules corresponding to each disease.

Alternatively, each of the one or more classification sub modules may predict a disease probability for a corresponding disease.

Alternatively, each of the one or more classification sub modules may derive a scalar value for predicting the disease probability.

Alternatively, each of the one or more classification sub modules may derive at least one of a probability value or a numerical value based on the derived scalar value.

Alternatively, the operations may further include preprocessing a length of the bio signal to correspond to an input length of the disease judgment model.

Alternatively, the preprocessing the length of the bio signal to correspond to the input length of the disease judgment model comprises at least one of: when the length of the bio signal exceeds the input length of the disease judgement model, deleting a portion of the bio signal that exceeds the input length of the disease judgment model; or when the length of the bio signal is less than the input length of the disease judgement model, matching the length of the bio signal to the input length of the disease judgment model by duplicating at least a portion of the bio signal.

Alternatively, the operations may further include: identifying a missing part in each of the one or more bio signals measured in the one or more leads; and making up for the missing part based on each of the one or more bio signals.

In order to solve one or more technical problems in the related art, there is disclosed a computer program judging a disease using a neural network, the computing device including: a processor with at least one core; and a memory comprising program codes executable by the processor, in which wherein the processor is configured to: acquire one or more bio signals respectively measured in one or more leads; and generate result information about a disease by inputting the one or more bio signals into a disease judgment model.

In order to solve one or more technical problems in the related art, there is disclosed a method of judging a disease using a neural network performed in a processor of a computing device, the method including: acquiring one or more bio signals respectively measured in one or more leads; and generating result information about a disease by inputting the one or more bio signals into a disease judgment model.

The present disclosure may provide the disease judgement method.

### BRIEF DESCRIPTION OF THE SEVERAL VIEWS OF THE DRAWINGS

FIG. 1 is a block diagram illustrating a computing device for judging a disease by using a neural network according to an embodiment of the present disclosure.

FIG. 2 is a diagram illustrating a process of judging a disease by using a neural network by the computing device according to the embodiment of the present disclosure.

FIG. 3 is a diagram illustrating a process of pre-processing a bio signal for judging a disease by using a neural network according to the embodiment of the present disclosure.

FIG. 4 is a schematic diagram illustrating a network function according to the embodiment of the present disclosure.

FIG. 5 is a flowchart illustrating the method of judging a disease by using a neural network by a processor according to the embodiment of the present disclosure.

FIG. 6 is a simple and general schematic diagram illustrating an example of a computing environment in which the embodiments of the present disclosure are implementable.

### DETAILED DESCRIPTION

Various embodiments are described with reference to the drawings. In the present specification, various descriptions are presented for understanding the present disclosure. However, it is obvious that the embodiments may be carried out even without a particular description.

Terms, “component,” “module,” “system,” and the like used in the present specification indicate a computer-related entity, hardware, firmware, software, a combination of software and hardware, or execution of software. For example, a component may be a procedure executed in a processor, a processor, an object, an execution thread, a program, and/or a computer, but is not limited thereto. For example, both an application executed in a computing device and a computing device may be components. One or more components may reside within a processor and/or an execution thread. One component may be localized within one computer. One component may be distributed between two or more computers. Further, the components may be executed by various computer readable media having various data structures stored therein. For example, components may communicate through local and/or remote processing according to a signal (for example, data transmitted to another system through a network, such as the Internet, through data and/or a signal from one component interacting with another component in a local system and a distributed system) having one or more data packets.

A term “or” intends to mean comprehensive “or” not exclusive “or.” That is, unless otherwise specified or when it is unclear in context, “X uses A or B” intends to mean one of the natural comprehensive substitutions. That is, when X uses A, X uses B, or X uses both A and B, “X uses A or B” may be applied to any one among the cases. Further, a term “and/or” used in the present specification shall be understood to designate and include all of the possible combinations of one or more items among the listed relevant items.

It should be understood that a term “include” and/or “including” means that a corresponding characteristic and/or a constituent element exists. Further, a term “include” and/or “including” means that a corresponding characteristic and/or a constituent element exists, but it shall be understood that the existence or an addition of one or more other characteristics, constituent elements, and/or a group thereof is not excluded. Further, unless otherwise specified or when it is unclear in context that a single form is indicated in context, the singular shall be construed to generally mean “one or more” in the present specification and the claims.

The term “at least one of A and B” should be interpreted to mean “the case including only A,” “the case including only B,” and “the case where A and B are combined.”

Those skilled in the art shall recognize that the various illustrative logical blocks, configurations, modules, circuits, means, logic, and algorithm operations described in relation to the embodiments additionally disclosed herein may be implemented by electronic hardware, computer software, or in a combination of electronic hardware and computer software. In order to clearly exemplify interchangeability of hardware and software, the various illustrative components, blocks, configurations, means, logic, modules, circuits, and operations have been generally described above in the functional aspects thereof. Whether the functionality is implemented as hardware or software depends on a specific application or design restraints given to the general system. Those skilled in the art may implement the functionality described by various methods for each of the specific applications. However, it shall not be construed that the determinations of the implementation deviate from the range of the contents of the present disclosure.

The description about the presented embodiments is provided so as for those skilled in the art to use or carry out the present disclosure. Various modifications of the embodiments will be apparent to those skilled in the art. General principles defined herein may be applied to other embodiments without departing from the scope of the present disclosure. Therefore, the present disclosure is not limited to the embodiments presented herein. The present disclosure shall be interpreted within the broadest meaning range consistent to the principles and new characteristics presented herein.

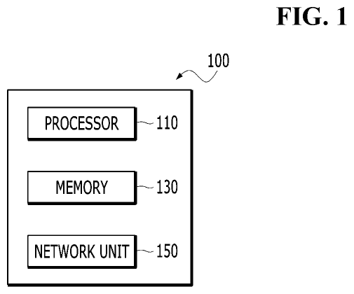

FIG. 1 is a block diagram illustrating a computing device for judging a disease by using a neural network according to an embodiment of the present disclosure.

The configuration of a computing device 100 illustrated in FIG. 1 is merely a simplified example. In the embodiment of the present disclosure, the computing device 100 may include other configurations for performing a computing environment of the computing device 100, and only some of the disclosed configurations may also configure the computing device 100.

The computing device 100 may include a processor 110, a memory 130, and a network unit 150.

The processor 110 may be formed of one or more cores, and may include a processor, such as a central processing unit (CPU), a general purpose graphics processing unit (GPGPU), and a tensor processing unit (TPU) of the computing device, for performing a data analysis and deep learning. The processor 110 may read a computer program stored in the memory 130 and process data for machine learning according to an embodiment of the present disclosure. According to the embodiment of the present disclosure, the processor 110 may perform calculation for training a neural network. The processor 110 may perform a calculation, such as processing of input data for training in Deep Learning (DL), extraction of a feature from input data, an error calculation, and updating of a weight of the neural network by using backpropagation, for training the neural network. At least one of the CPU, GPGPU, and TPU of the processor 110 may process training of a network function. For example, the CPU and the GPGPU may process training of the network function and data classification by using a network function together. Further, in the embodiment of the present disclosure, the training of the network function and the data classification by using a network function may be processed by using the processors of the plurality of computing devices together. Further, the computer program executed in the computing device according to the embodiment of the present disclosure may be a CPU, GPGPU, or TPU executable program.

According to the embodiment of the present disclosure, the memory 130 may store a predetermined (or selected) type of information generated or determined by the processor 110 and a predetermined (or selected) type of information received by a network unit 150.

According to the embodiment of the present disclosure, the memory 130 may include at least one type of storage medium among a flash memory type, a hard disk type, a multimedia card micro type, a card type of memory (for example, an SD or XD memory), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk. The computing device 100 may also be operated in relation to web storage performing a storage function of the memory 130 on the Internet. The description of the foregoing memory is merely illustrative, and the present disclosure is not limited thereto. The network unit 150 according to several embodiments of the present disclosure may use various wired communication systems, such as a Public Switched Telephone Network (PSTN), an x Digital Subscriber Line (xDSL), a Rate Adaptive DSL (RADSL), a Multi Rate DSL (MDSL), a Very High Speed DSL (VDSL), a Universal Asymmetric DSL (UADSL), a High Bit Rate DSL (HDSL), and a local area network (LAN).

The network unit 150 presented in the present specification may use various wireless communication systems, such as Code Division Multi Access (CDMA), Time Division Multi Access (TDMA), Frequency Division Multi Access (FDMA), Orthogonal Frequency Division Multi Access (OFDMA), Single Carrier-FDMA (SC-FDMA), and other systems.

The network unit 150 in the present disclosure may be configured regardless of its communication mode, such as a wired mode and a wireless mode, and may be configured of various communication networks, such as a Personal Area Network (PAN) and a Wide Area Network (WAN). Further, the network may be the publicly known World Wide Web (WWW), and may also use a wireless transmission technology used in the short range communication, such as Infrared Data Association (IrDA) or Bluetooth.

The technologies described in the present specification may be used in other networks, as well as the foregoing networks.

In the embodiment of the present disclosure, the processor 110 may acquire one or more bio signals measured in each of one or more leads. The processor 110 may acquire a bio signal measured from a lead of a separate measuring device, or may also directly acquire a bio signal from the lead.

The processor 110 may pre-process a length of a bio signal so as to correspond to an input length of a disease judgement model 200.

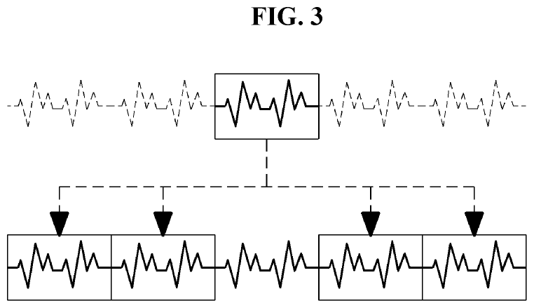

Herein, the pre-processing process will be described below with reference to FIG. 3.

The processor 110 may input one or more bio signals to the disease judgement model 200 and generate result information about a disease.

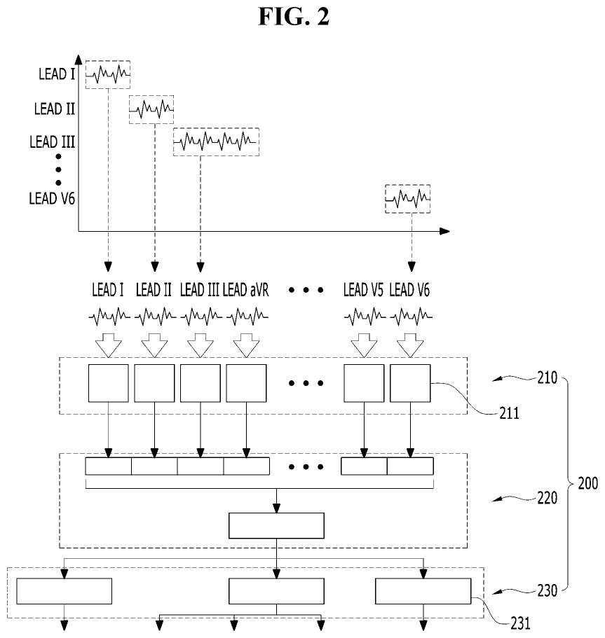

Herein, the disease judgement model 200 will be described below with reference to FIG. 2.

FIG. 2 is a diagram illustrating a process of judging a disease by using a neural network by the computing device according to the embodiment of the present disclosure.

The disease judgement model 200 may include an encoding module 210, a concentration module 220, and a classification module 230.

The encoding module 210 may extract a feature from the bio signal. The encoding module 210 may include one or more encoding sub modules 211 formed in the number corresponding to the input channel of the bio signal. Herein, each of one or more encoding sub modules 211 may be formed of a plurality of blocks performing an encoding operation, and one or more blocks may include a skip connection.

In the blocks, convolution and pooling occur once in the first block, and convolution and pooling may be repeated several times from the second block to the last block. Further, the pooling may include max pooling that extracts a maximum value from a partial area of data, and an average pooling that calculates an average value of each area for a partial area of data.

Each of one or more encoding sub modules 211 may share at least some of a weight.

Herein, each encoding sub module 211 may also be a module having the same structure that shares a weight with each other. The disease judgement model 200 may extract a feature by encoding each bio signal measured from the plurality of leads by using the same encoding sub module 211.

The concatenation module 220 may generate concatenation encoding data by concatenating one or more data encoded by the encoding module 210. The concatenation module 220 may process the plurality of encoded data output from the plurality of encoding modules to the form processible by the classification module 230.

The classification module 230 may receive the concatenation encoding data and generate result information. Herein, the classification module 230 may include a classification sub module 231 corresponding to each disease. Further, one classification sub module 231 may predict at least one disease. Further, each classification sub module 231 may predict a disease probability for each disease.

Each classification sub module 231 may also derive a scalar value related to the disease. For example, each classification sub module 231 may also derive a scalar value (for example, blood pressure) for a specific item of a bio signal related to a disease. The foregoing description is merely illustrative, and the present disclosure is not limited thereto. FIG. 3 is a diagram illustrating a process of pre-processing a bio signal for judging a disease by using a neural network according to the embodiment of the present disclosure.

When a length of a bio signal is smaller than an input length of the disease judgement model 200, the processor 110 may match the length of the bio signal to the input length of the disease judgement model 200 by copying at least a part of the bio signal.

Otherwise, when the processor 110 performs the pre-processing so that the length of the bio signal corresponds to the input length of the disease judgement model 200, and the length of the bio signal is larger than the input length of the disease judgement model 200, the processor 110 may remove a part exceeding the input length of the disease judgement model 200 from the bio signal.

The processor 110 may identify a bio signal missing part in each bio signal measured from one or more leads. Further, the processor 110 may make up the bio signal missing part based on each bio signal. For example, the bio signal missing part may be supplemented by copying the bio signal of the corresponding lead measured at a different time.

For another example, the bio signal missing part may also be generated through the neural network model trained with the measured bio signal as training data.

When the bio signal is a one-dimensional signal, the processor 110 may convert the bio signal into a signal in a frequency domain. For example, the processor 110 may convert the bio signal acquired with a function of a time domain into a frequency function by performing Fourier transformation. When the bio signals have different frequencies, the processor 110 may arbitrarily match the frequencies by applying interpolation, for example, bicubic and spline interpolation. For example, in the case of an electrocardiogram bio signal, the processor 110 may obtain a signal reconstructed at the same interval by calculating time-series X-coordinates of the RR intervals, which are the intervals from the R wave to the R wave, calculating y-coordinates representing the change of the RR interval corresponding to the time series, performing interpolation between the coordinates by spline interpolation, sampling the coordinates again at a constant frequency. Herein, the spline interpolation method is the method of obtaining an interpolation function by using a spline function, which is a connection polynomial applying a low-order polynomial to a subset of points to make a smooth connection. The foregoing interpolation method is merely an example, and the present disclosure is not limited thereto.

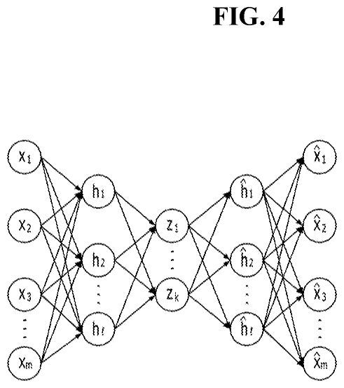

FIG. 4 is a schematic diagram illustrating a network function according to the embodiment of the present disclosure.

Throughout this specification, the deep learning based model, the computation model, the neural network, a network function, and the neural network may be used as the same meaning. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship between an input node and an output node. Concepts of the input node and the output node are relative and a predetermined (or selected) node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired or a selected function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, definition of the layer is predetermined (or selected) for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean not the initial input node and the final output node but the nodes constituting the neural network.

In the neural network according to an embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another embodiment of the present disclosure, the number of nodes of the input layer may be larger than the number of nodes of the output layer, and the neural network may be a neural network of a type in which the number of nodes increases from the input layer to the hidden layer. The neural network according to still yet another embodiment of the present disclosure may be a neural network of a type in which the neural networks are combined.

A deep neural network (DNN) may refer to a neural network that includes a plurality of hidden layers in addition to the input and output layers. When the deep neural network is used, the latent structures of data may be determined. That is, potential structures of photos, text, video, voice, and music (e.g., what objects are in the picture, what the content and feelings of the text are, what the content and feelings of the voice are) may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, a Generative Adversarial Network (GAN), and the like. The description of the deep neural network described above is just an example and the present disclosure is not limited thereto.

In an embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be learned in at least one scheme of supervised learning, unsupervised learning, semi supervised learning, or reinforcement learning. The learning of the neural network may be a process in which the neural network applies knowledge for performing a specific operation to the neural network.

The neural network may be learned in a direction to reduce or minimize errors of an output. The learning of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weight of each node of the neural network. In the case of the supervised learning, the learning data labeled with a correct answer is used for each learning data (e.g., the labeled learning data) and in the case of the unsupervised learning, the correct answer may not be labeled in each learning data. That is, for example, the learning data in the case of the supervised learning related to the data classification may be data in which category is labeled in each learning data. The labeled learning data is input to the neural network, and the error may be calculated by comparing the output (category) of the neural network with the label of the learning data. As another example, in the case of the unsupervised learning related to the data classification, the learning data as the input is compared with the output of the neural network to calculate the error. The calculated error is back-propagated in a reverse direction (e.g., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the learning data may be generally a subset of actual data (e.g., data to be processed using the learned neural network) of actual data, and as a result, there may be a learning cycle in which errors for the learning data decrease, but the errors for the actual data increase. Overfitting is a phenomenon in which the errors for the actual data increase due to excessive learning of the learning data. For example, a phenomenon in which the neural network that learns a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the learning data, regularization, dropout of omitting a part of the node of the network in the process of learning, utilization of a batch normalization layer, etc., may be applied.

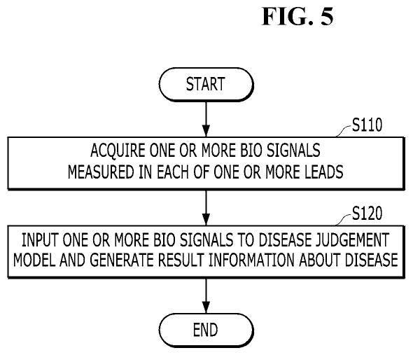

FIG. 5 is a flowchart illustrating the method of judging a disease by using a neural network by a processor according to the embodiment of the present disclosure.

The processor 110 according to the present disclosure may acquire one or more bio signals measured from each of one or more leads (S110).

The processor 110 may input one or more bio signals to the disease judgement model 200 and generate result information about the disease (S120).

Herein, the detailed descriptions of the processor 110 and the disease judgement model 200 may be replaced with the contents described above with reference to FIGS. 1 to 4, and each operation may be omitted or added based on the foregoing contents.

### Experimental Example 1

Accuracy of the electrocardiogram measurement method (“Prediction of mortality from 12-lead electrocardiogram voltage data using a deep neural network”, Nature Medicine volume 26, pages 886-891, 2020.05.11.) according to the related art and the disease judgement method according to the embodiment of the present disclosure was measured by using overall measurement data of hospital A, overall measurement data of hospital B, and clinical data of hospital B.

Herein, the data may further include information about a patient. The information about the patient may include, for example, age, gender, health checkup information, and questionnaire information of the patient. For example, the health checkup information may be data, such as blood sugar and blood pressure, obtained through body measurement, and the questionnaire information may be data, such as exercise amount, obtained through Q&A on the patient. The foregoing data is merely an example, and the present disclosure is not limited thereto.

The electrocardiogram measurement method according to the related art encodes an electrocardiogram signal received through a plurality of input channels at once, and differently from this, in the disease judgement method according to the embodiment of the present disclosure, the encoding sub module 211 is configured in a number corresponding to the input channel and encodes the measured bio signal for each channel.

Herein, the accuracy was measured through an Area Under a Receiver Operating Characteristic (ROC) curve (AUROC).

In the ROC curve, an x-axis represents a False Positive Rate (FPR), and a y-axis represents a True Positive Rate (TPR), and the ROC curve is in the range of [0,1] on the xi-axis and y-axis, and is a curve connecting from (0,0) to (1,1).

Herein, the FPR is the value obtained by subtracting specificity from 1, and specificity is the rate at which a normal case is judged normal. That is, specificity is the rate at which people who are normal for a specific disease are judged negative for the specific disease. Accordingly, the FPR may be considered as the rate at which people who are normal for the specific disease are judged positive for the specific disease.

The TPR represents sensitivity, and the sensitivity is the rate at which an abnormal case is judged abnormal. That is, the TPR is the rate at which people with a certain disease are judged positive.

That is, in the ROC curve, it is possible to know the degree of increase in sensitivity according to the degree of decrease in specificity, and as the area under the ROC curve is closer to the maximum value of 1, the accuracy is higher.

Table 1 below is the table representing the result of the comparison between the accuracy of the electrocardiogram measurement method according to the related art and the accuracy of the disease judgement method according to the embodiment of the present disclosure.

TABLE 1

| | Overall | Overall | |
|---|---|---|---|
| | measurement | measurement | Clinical |
| | data of | data of | data of |
| | hospital A | hospital B | hospital B |
| Electrocardiogram | 0.927 | 0.909 | 0.889 |
| measurement | | | |
| method according | | | |
| to the related art | | | |
| Disease judgement | 0.934 | 0.915 | 0.909 |
| method according | | | |
| to the embodiment | | | |
| of the present | | | |
| disclosure | | | |

As a result of the measurement, the electrocardiogram measurement method according to the related art showed accuracy of 0.927 for the overall measurement data of hospital A, accuracy of 0.909 for the overall measurement data of hospital B, and accuracy of 0.889 for the clinical data of hospital B.

The disease judgement method according to the embodiment of the present disclosure showed accuracy of 0.934 for the overall measurement data of hospital A, accuracy of 0.915 for the overall measurement data of hospital B, and accuracy of 0.909 for the clinical data of hospital B.

As the result of the comparison of the accuracy, it can be seen that the disease judgement method according to the embodiment of the present disclosure shows higher accuracy for all data than the electrocardiogram measurement method according to the related art.

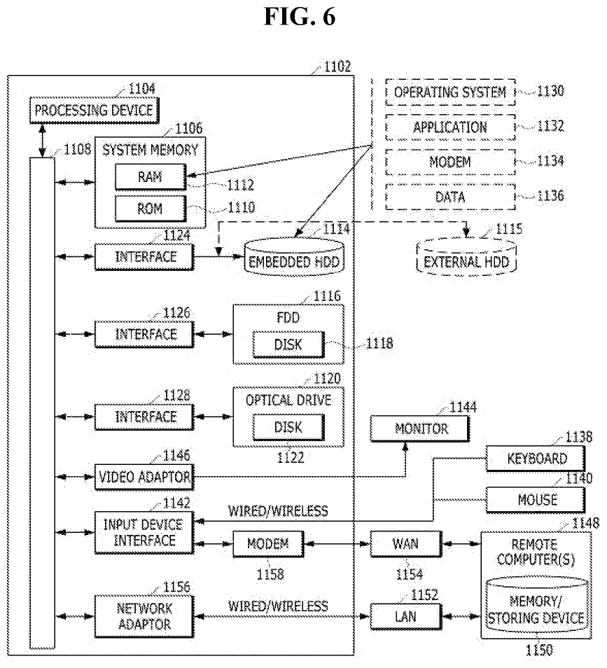

FIG. 6 is a simple and normal schematic view of a computing environment in which the embodiments of the present disclosure may be implemented.

It is described above that the present disclosure may be generally implemented by the computing device, but those skilled in the art will well know that the present disclosure may be implemented in association with a computer executable command which may be executed on one or more computers and/or in combination with other program modules and/or as a combination of hardware and software.

In general, the program module includes a routine, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devices as well as a single-processor or multi-processor computer system, a mini computer, and a main frame computer.

The embodiments described in the present disclosure may also be implemented in a distributed computing environment in which predetermined (or selected) tasks are performed by remote processing devices connected through a communication network. In the distributed computing environment, the program module may be positioned in both local and remote memory storage devices.

The computer generally includes various computer readable media. Media accessible by the computer may be computer readable media regardless of types thereof and the computer readable media include volatile and non-volatile media, transitory and non-transitory media, and mobile and non-mobile media. As a non-limiting example, the computer readable media may include both computer readable storage media and computer readable transmission media. The computer readable storage media include volatile and non-volatile media, temporary and non-temporary media, and movable and non-movable media implemented by a predetermined (or selected) method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined (or selected) other media which may be accessed by the computer or may be used to store desired information, but are not limited thereto.

The computer readable transmission media generally implement the computer readable command, the data structure, the program module, or other data in a carrier wave or a modulated data signal such as other transport mechanism and include all information transfer media. The term “modulated data signal” means a signal acquired by configuring or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined (or selected) processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an interior hard disk drive (HDD) 1114 (for example, EIDE and SATA), in which the interior hard disk drive 1114 may also be configured for an exterior purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD, and the like). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. An interface 1124 for implementing an exterior drive includes at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined (or selected) data in an appropriate digital format. In the description of the computer readable media, the mobile optical media such as the HDD, the mobile magnetic disk, and the CD or the DVD are mentioned, but it will be well appreciated by those skilled in the art that other types of media readable by the computer such as a zip drive, a magnetic cassette, a flash memory card, a cartridge, and others may also be used in an operating environment and further, the predetermined (or selected) media may include computer executable commands for executing the methods of the present disclosure.

Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a computing device computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158 or has other means that configure communication through the WAN 1154 such as connection to a communication computing device on the WAN 1154 or connection through the Internet. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is and other means configuring a communication link among computers may be used.

The computer 1102 performs an operation of communicating with predetermined (or selected) wireless devices or entities which are disposed and operated by the wireless communication, for example, the printer, a scanner, a desktop and/or a portable computer, a portable data assistant (PDA), a communication satellite, predetermined (or selected) equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11(a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It will be appreciated by those skilled in the art that information and signals may be expressed by using various different predetermined (or selected) technologies and techniques. For example, data, instructions, commands, information, signals, bits, symbols, and chips which may be referred in the above description may be expressed by voltages, currents, electromagnetic waves, magnetic fields or particles, optical fields or particles, or predetermined (or selected) combinations thereof. It may be appreciated by those skilled in the art that various logical blocks, modules, processors, means, circuits, and algorithm steps described in association with the embodiments disclosed herein may be implemented by electronic hardware, various types of programs or design codes (for easy description, herein, designated as software), or a combination of all of them. In order to clearly describe the intercompatibility of the hardware and the software, various components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various embodiments presented herein may be implemented as manufactured articles using a method, an apparatus, or a standard programming and/or engineering technique. The term manufactured article includes a computer program, a carrier, or a medium which is accessible by a predetermined (or selected) computer-readable storage device. For example, a computer-readable storage medium includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. Further, various storage media presented herein include one or more devices and/or other machine-readable media for storing information.

It will be appreciated that a specific order or a hierarchical structure of steps in the presented processes is one example of accesses. It will be appreciated that the specific order or the hierarchical structure of the steps in the processes within the scope of the present disclosure may be rearranged based on design priorities. Appended method claims provide elements of various steps in a sample order, but the method claims are not limited to the presented specific order or hierarchical structure.

The description of the presented embodiments is provided so that those skilled in the art of the present disclosure use or implement the present disclosure. Various modifications of the embodiments will be apparent to those skilled in the art and general principles defined herein can be applied to other embodiments without departing from the scope of the present disclosure. Therefore, the present disclosure is not limited to the embodiments presented herein, but should be interpreted within the widest range which is coherent with the principles and new features presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A computer program stored in a computer readable storage medium, wherein when the computer program is executed on at least one processor, the computer program causes the processor to perform the following operations for judging a disease using a neural network, the operations comprising:
acquiring one or more bio signals respectively measured in one or more leads;
inputting the one or more bio signals into a disease judgment model; and
generating result information about a disease in response to inputting the one or more bio signals into the disease judgment model.

2. The computer program stored in a computer readable storage medium of claim 1, wherein the disease judgment model comprising:
an encoding module comprising one or more encoding sub modules;
a concatenation module for generating concatenation encoding data by concatenating one or more data encoded by the encoding module; and
a classification module that receives the concatenation encoding data and generates the result information.

3. The computer program stored in a computer readable storage medium of claim 2, wherein each of the one or more encoding sub modules comprises a plurality of blocks that perform an encoding operation, and
at least one block of the plurality of blocks comprises a skip connection.

4. The computer program stored in a computer readable storage medium of claim 2, wherein each of the one or more encoding sub modules shares at least some of weight.

5. The computer program stored in a computer readable storage medium of claim 2, wherein the classification module comprises one or more classification sub modules corresponding to each disease.

6. The computer program stored in a computer readable storage medium of claim 5, wherein each of the one or more classification sub modules predicts a disease probability for a corresponding disease.

7. The computer program stored in a computer readable storage medium of claim 6, wherein each of the one or more classification sub modules derives a scalar value for predicting the disease probability.

8. The computer program stored in a computer readable storage medium of claim 7, wherein each of the one or more classification sub modules derives at least one of a probability value or a numerical value based on the derived scalar value.

9. The computer program stored in a computer readable storage medium of claim 1, wherein the operations further comprising:
preprocessing a length of the bio signal to correspond to an input length of the disease judgment model.

10. The computer program stored in a computer readable storage medium of claim 9, wherein the preprocessing the length of the bio signal to correspond to the input length of the disease judgment model comprises at least one of:
when the length of the bio signal exceeds the input length of the disease judgement model, deleting a portion of the bio signal that exceeds the input length of the disease judgment model; or
when the length of the bio signal is less than the input length of the disease judgement model, matching the length of the bio signal to the input length of the disease judgment model by duplicating at least a portion of the bio signal.

11. The computer program stored in a computer readable storage medium of claim 1, wherein the operations further comprising:
identifying a missing part in each of the one or more bio signals measured in the one or more leads; and
making up for the missing part based on each of the one or more bio signals.

12. A computing device judging a disease using a neural network, the computing device comprising:
a processor with at least one core; and
a memory comprising program codes executable by the processor,
wherein the processor is configured to:
acquire one or more bio signals respectively measured in one or more leads; and
generate result information about a disease by inputting the one or more bio signals into a disease judgment model.

13. A method of judging a disease using a neural network performed in a processor of a computing device, the method comprising:
acquiring one or more bio signals respectively measured in one or more leads; and
generating result information about a disease by inputting the one or more bio signals into a disease judgment model.
